(19) United States
(12) Patent Application Publication
(10) Pub. No.: US 2026/0224804 A1
(43) Pub. Date: Aug. 6, 2026

(54) INTRAVENOUS PUMP BACKPACK DEVICE

(71) Applicant: Savannah Thompson, Palestine, TX (US)

(72) Inventor: Savannah Thompson, Palestine, TX (US)

(21) Appl. No.: 19/047,081

(22) Filed: Feb. 6, 2025

(51) Int. Cl. A61M 5/142; A45F 3/00; A45F 3/04

(52) U.S. Cl. CPC A61M 5/14244; A45F 3/04; A45F 2003/003; A61M 2209/06; A61M 2209/088

(57) ABSTRACT

An intravenous pump backpack device includes a body harness worn around a body of the user and a panel which is coupled to the body harness. The panel is supported on a back of the user when the user is wearing the body harness. The panel has an opening extending through the panel. A storage pocket is coupled to the panel to store an intravenous pump. The storage pocket has a hole which is aligned with the opening in the panel such that the intravenous pump is accessible through the opening. A storage pouch is removably attachable to the panel to contain a bag of intravenous medication. The storage pouch has an access opening which is aligned with the opening in the panel such that the bag of intravenous medication can be connected to the intravenous pump.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) the Names of the Parties to a Joint Research Agreement

Not Applicable

### (e) Incorporation-by-reference of Material Submitted on a

### COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to backpack devices and more particularly pertains to a new backpack device for carrying an intravenous pump and a bag of intravenous medication on a user. The device includes a body harness that is wearable on a body of a user and a panel that is attached to the body harness. The device includes a storage pocket that is coupled to the panel which stores an intravenous pump and a storage pouch that is removably attachable to the panel which stores a bag of intravenous medication.

### (2) Description of Related Art Including Information Disclosed Under 37 CFR 1.97 and 1.98

The prior art relates to backpack devices including: a vest and backpack device that includes a vest that is wearable on a child and a backpack coupled to the vest and a milk pouch integrated into the backpack and a pump pocket integrated into the backpack; a intravenous fluid delivery device that includes an intravenous bag that has a plurality of channels for evenly distributing an intravenous medication within the intravenous bag when the intravenous bag is wrapped around a shoulder of a user and a pump integrated into the intravenous bag; an intravenous backpack device that includes a plurality of straps that can be wrapped around a shoulder of a user and a plurality of clasps which are removably attachable to the plurality of straps and an intravenous container that is removably attachable to the plurality of clasps; an intravenous backpack device that includes a backpack and a support rod which extends upwardly from the backpack to support an intravenous bag. In no instance does the prior art disclose a backpack device that includes a body harness and a panel attached to the body harness and a storage pocket coupled to the body harness for storing an intravenous pump and a storage pouch that is removably attachable to an opposite side of the panel from the storage pocket to store a bag of intravenous medication.

### (h) BRIEF SUMMARY OF THE INVENTION

An embodiment of the disclosure meets the needs presented above by generally comprising a body harness worn around a body of the user and a panel which is coupled to the body harness. The panel is supported on a back of the user when the user is wearing the body harness. The panel has an opening extending through the panel. A storage pocket is coupled to the panel to store an intravenous pump. The storage pocket has a hole which is aligned with the opening in the panel such that the intravenous pump is accessible through the opening. A storage pouch is removably attachable to the panel to contain a bag of intravenous medication. The storage pouch has an access opening which is aligned with the opening in the panel such that the bag of intravenous medication can be connected to the intravenous pump.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a back view of an intravenous pump backpack device according to an embodiment of the disclosure.

FIG. 2 is a left side view of an embodiment of the disclosure.

FIG. 3 is a back view of an embodiment of the disclosure showing a flap portion of a storage pocket in an open position.

FIG. 4 is a back view of an embodiment of the disclosure showing a cover being detached from a storage pocket.

FIG. 5 is a front view of an embodiment of the disclosure.

FIG. 6 is a front exploded view of an embodiment of the disclosure showing a storage pouch being detached from a panel.

FIG. 7 is a perspective view of a storage pouch and a bag of intravenous medication of an embodiment of the disclosure.

FIG. 8 is a perspective in-use view of an embodiment of the disclosure.

### (j) DETAILED DESCRIPTION OF THE INVENTION

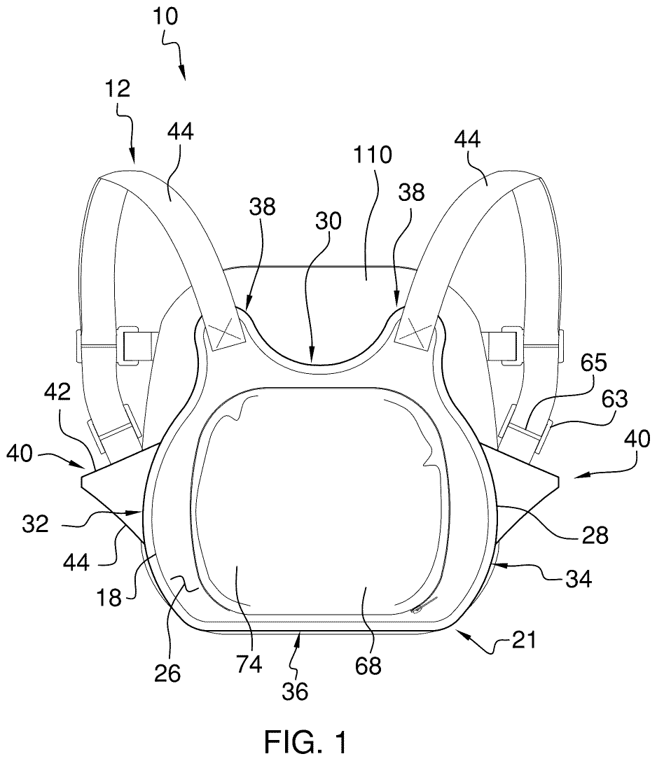

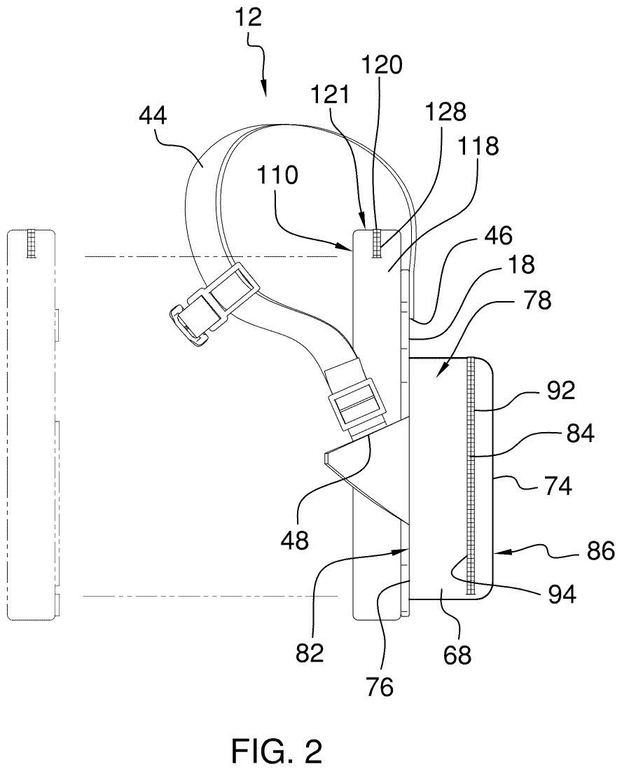

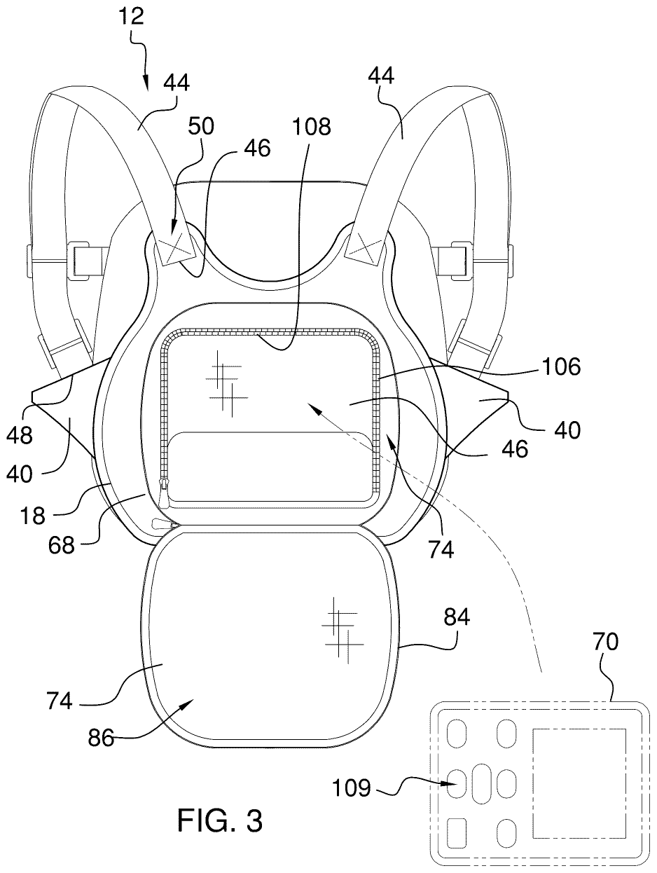

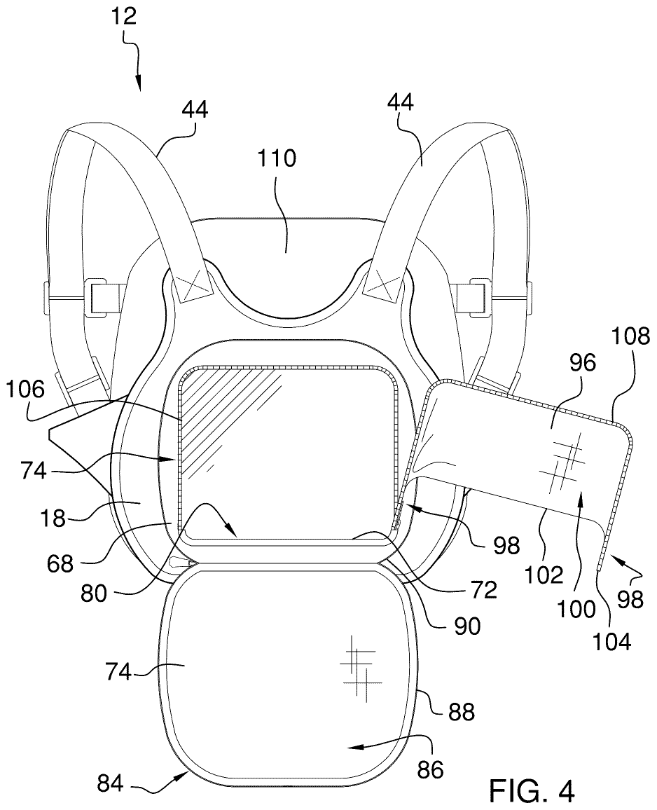

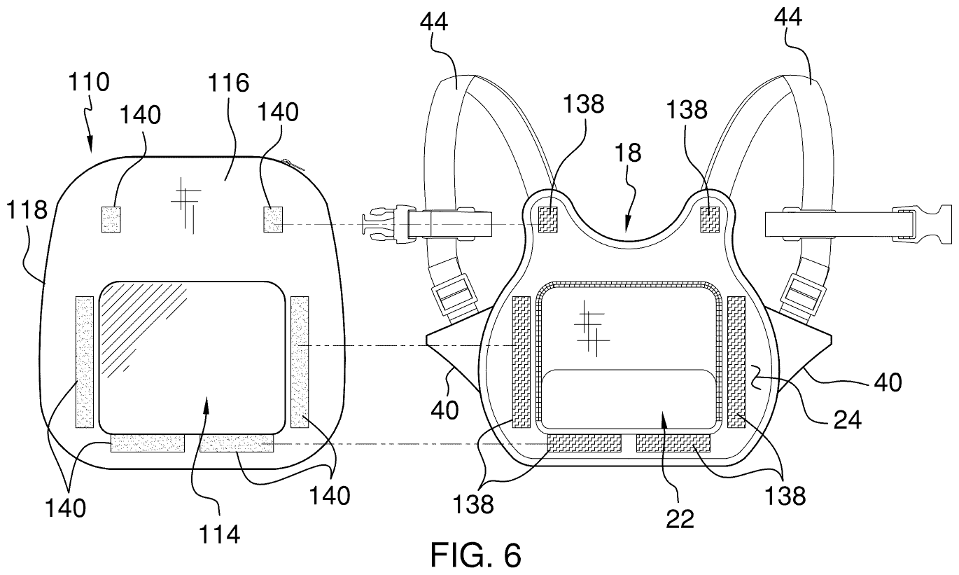

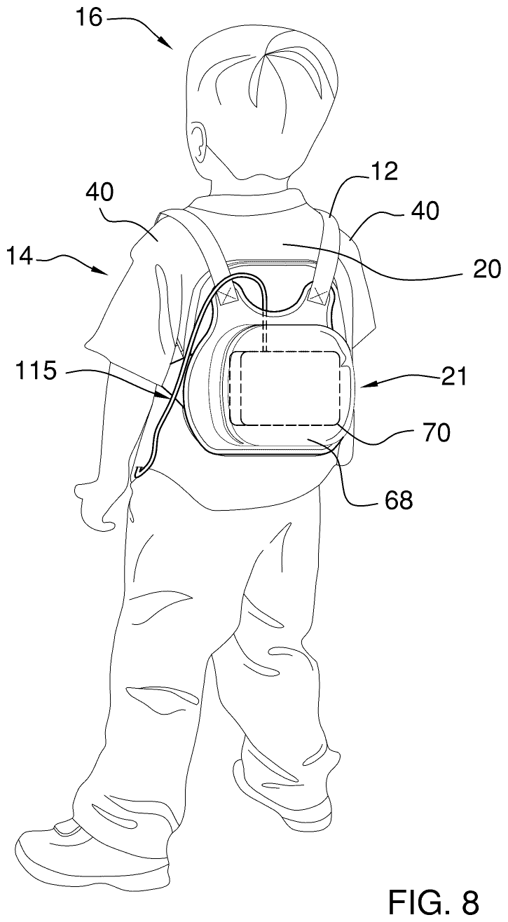

With reference now to the drawings, and in particular to FIGS. 1 through 8 thereof, a new backpack device embodying the principles and concepts of an embodiment of the disclosure and generally designated by the reference numeral 10 will be described.

As best illustrated in FIGS. 1 through 8, the intravenous pump backpack device 10 generally comprises a body harness 12 that can be worn around a body 14 of the user 16. The user 16 may be a child who is undergoing blinatumomab treatment for leukemia, for example, or other medical situation that involves the regular administration of intravenous medication over an extended period of time. Furthermore, the user 16 may be a toddler under the age of five or an infant. A panel 18 is coupled to the body harness 12 such that the panel 18 is supported on a back 20 of the user 16 when the user 16 is wearing the body harness 12. The panel 18 and the body harness 12 define a backpack 21 that is wearable on the user 16. Additionally, the panel 18 has an opening 22 extending through the panel 18.

The panel 18 has a front surface 24 and a back surface 26 and a perimeter edge 28 extending between the front surface 24 and the back surface 26; the perimeter edge 28 has a top side 30 and a first lateral side 32 and a second lateral side 34 and a bottom side 36. The perimeter edge 28 has a pair of lobe sections 38 and each lobe section 38 of the pair of lobe sections 38 extends upwardly from the top side 30. Furthermore, each lobe section 38 of the pair of lobe sections 38 extends toward a respective shoulder 40 of the user 16 when the user 16 is wearing the backpack. Each lobe section 38 of the pair of lobe sections 38 is aligned with a respective one of the first lateral side 32 and the second lateral side 34. Each of the first lateral side 32 and the second lateral side 34 curve outwardly between the top side 30 and the bottom side 36 such that the panel 18 extends across a full width of the back 20 of the user 16 when the user 16 is wearing the body harness 12.

The opening 22 extends between the front surface 24 and the back surface 26; the opening 22 is positioned closer to the bottom side 36 than the top side 30. The panel 18 has a pair of flaps 40 each coupled to and extending laterally away from a respective one of the first lateral side 32 and the second lateral side 34 of the perimeter edge 28. Furthermore, each flap of the pair of flaps 40 extends around the body 14 of the user 16 when the user 16 is wearing the body harness 12. Additionally, each flap 40 of the pair of flaps 40 has an upper edge 42 which is oriented at an angle with a bottom edge 43.

The body harness 12 includes a pair of shoulder straps 44 and each shoulder strap 44 of the pair of shoulder straps 44 has a first end 46 and a second end 48. Each shoulder strap 44 of the pair of shoulder straps 44 has a first connection point 50 which is located adjacent to the first end 46 of a respective shoulder strap 44. Each shoulder strap 44 of the pair of shoulder straps 44 is attached to a respective lobe section 38 of the pair of lobe sections 38 of the perimeter edge 28 of the panel 18 at the first connection point 50. Furthermore, the second end 48 of each shoulder strap 44 of the pair of shoulder straps 44 is coupled to the upper edge 42 of a respective one of the pair of flaps 40 of the panel 18. Each shoulder strap 44 of the pair of shoulder straps 44 may comprise a first half 52 and a second half 54 and an adjustment slide 56 which is attached to the first half 52 and through which the second half 54 slides to adjust a length of each of the shoulder straps 44.

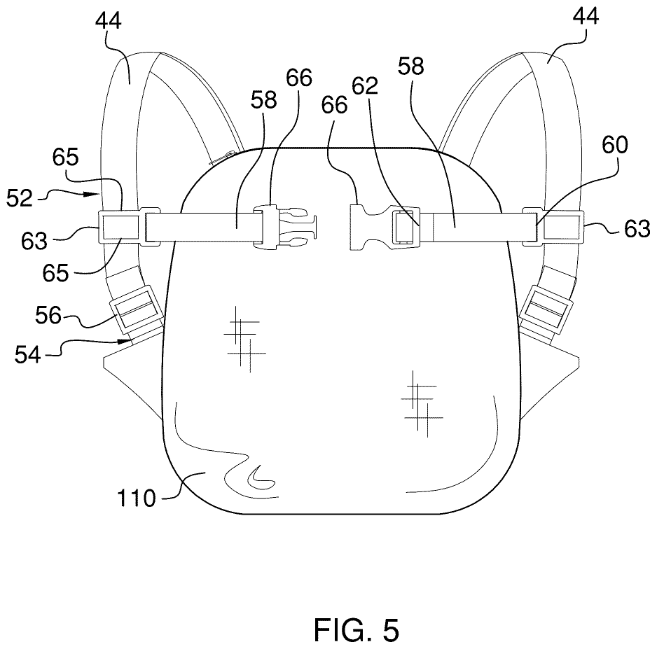

The body harness 12 includes a pair of chest straps 58 and each chest strap 58 of the pair of chest straps 58 has a coupled end 60 and a free end 62. The coupled end 60 of each chest strap 58 is movably attached to a respective one of the pair of shoulder straps 44 such that the pair of chest straps 58 can be slid upwardly or downwardly on the respective shoulder strap 44 to adjust fitment on the user 16. Furthermore, each chest strap 58 of the pair of chest straps 58 extends across a chest 64 of the user 16 when the user 16 is wearing the body harness 12. The free end 62 of the pair of chest straps 58 is releasably matable to each other to inhibit the pair of shoulder straps 44 from sliding sideways off of the respective shoulder 40 of the user 16. A pair of buckles 66, or other type of quick release coupler commonly associated with strapping devices, is each coupled to the free end 62 of a respective chest strap 58 and the pair of buckles 66 is releasably matable to each other to connect the chest straps 58 together. As is shown in FIGS. 1 and 5, a pair of strap couplers 63 is each attached to the coupled end 60 of a respective chest strap 58 and each strap coupler 63 includes a plurality of members 65 through which a respective shoulder strap 44 is interlaced such that the pair of strap couplers 63 is slidable along the respective shoulder strap 44.

A storage pocket 68 is coupled to the panel 18 for storing an intravenous pump 70. The intravenous pump 70 may be an electronic intravenous pump that is prescribed by a physician for the administration of intravenous medication over an extended duration of time. The storage pocket 68 has a hole 72 which is aligned with the opening 22 in the panel 18. Furthermore, the intravenous pump 70 is accessible through the opening 22 and the hole 72 when the intravenous pump 70 is being stored in the storage pocket 68. The storage pocket 68 has a rear wall 74 and a front wall 76 and an outer wall 78 extending between the rear wall 74 and the front wall 76. The hole 72 extends through the front wall 76 and the hole 72 has a bounding edge 80. The front wall 76 has a forward surface 82 and the forward surface 82 is coupled to the back surface 26 of the panel 18.

The outer wall 78 has a cut 84 extending through the outer wall 78 and the cut 84 extends around a partial perimeter of the outer wall 78; the cut 84 defines a flap portion 86 of the storage pocket 68. The flap portion 86 is positionable in a closed position to retain the intravenous pump 70 in the storage pocket 68. Conversely, the flap portion 86 is positionable in an open position to remove or install the intravenous pump 70 from the storage pocket 68. The cut 84 has a first edge 88 and a second edge 90. A first closure 92 is coupled to the first edge 88 and the first closure 92 extending along a full length of the first edge 88. A second closure 94 is coupled to the second edge 90 of the cut 84 in the outer wall 78 and the second closure 94 extends along a full length of the second edge 90. The second closure 94 is releasably matable to the first closure 92 for securing the pocket in the closed position. Additionally, each of the first closure 92 and the second closure 94 may comprise a zipper or other type of multiple use, mechanical closure.

A cover 96 is provided and the cover 96 has a pair of leg portions 98 each extending downwardly from a body portion 100 of the cover 96. The body portion 100 has a lower edge 102 and the lower edge 102 is spaced upwardly from a distal end 104 of each leg portion 98 of the pair of leg portions 98. The cover 96 is comprised of a translucent material, including but not being limited vinyl or other translucent and flexible material, such that the intravenous pump 70 is visible through the cover 96. A third closure 106 is coupled to the bounding edge 80 of the hole 72 in the front wall 76 of the storage pocket 68 and the third closure 106 extends around a partial perimeter of the bounding edge 80.

A fourth closure 108 is coupled to the cover 96 and the fourth closure 108 extends over the body portion 100 of the cover 96 and downwardly along each leg portion 98 of the pair of leg portions 98 of the cover 96. The fourth closure 108 is releasably matable to the third closure 106 and each of the third closure 106 and the fourth closure 108 may comprise a zipper or other type of multiple use, mechanical coupler. The lower edge 102 of the body portion 100 is spaced upwardly from the bounding edge 80 of the hole 72 in the rear wall 74 of the storage pocket 68 when the fourth closure 108 is releasably mated to the third closure 106. Furthermore, the cover 96 secures the intravenous pump 70 in the storage pocket 68 such that controls 109 of the intravenous pump 70 are positioned beneath the body portion 100 of the cover 96 having the controls 109 being exposed in the hole 72.

A storage pouch 110 is provided and the storage pouch 110 is removably attachable to the panel 18. The storage pouch 110 stores a bag of intravenous medication 112 such that the bag of intravenous medication 112 is stored in proximity to the intravenous pump 70 when the storage pouch 110 is removably attached to the panel 18. The storage pouch 110 has an access opening 114 to access an interior of the storage pouch 110. The access opening 114 is aligned with the opening 22 in the panel 18 when the storage pouch 110 is removably attached to the panel 18. Furthermore, an intravenous line 115 can be extended through the access opening 114 such that the intravenous line 115 can extend between the user 16 and the intravenous pump 70 to deliver intravenous medication to the user 16.

The storage pouch 110 has a back wall 116 and an outside wall 118; the outside wall 118 has a cut 120 in the outside wall 118 which extends into an interior of the storage pouch 110. The cut 120 in the outside wall 118 enables the bag of intravenous medication 112 to pass into or out of the storage pouch 110. The outside wall 118 has an upper side 121 and a first lateral side 122 of the outside wall 118 and a second lateral side 123 of the outside wall 118. The cut 120 in the outside wall 118 extends along a full length of the upper side 121 and downwardly along each of the first lateral side 122 of the outside wall 118 and the second lateral side 123 of the outside wall 118. Additionally, the cut 120 in the outside wall 118 has a primary edge 124 and a secondary edge 125 and the back wall 116 has a rear surface 126.

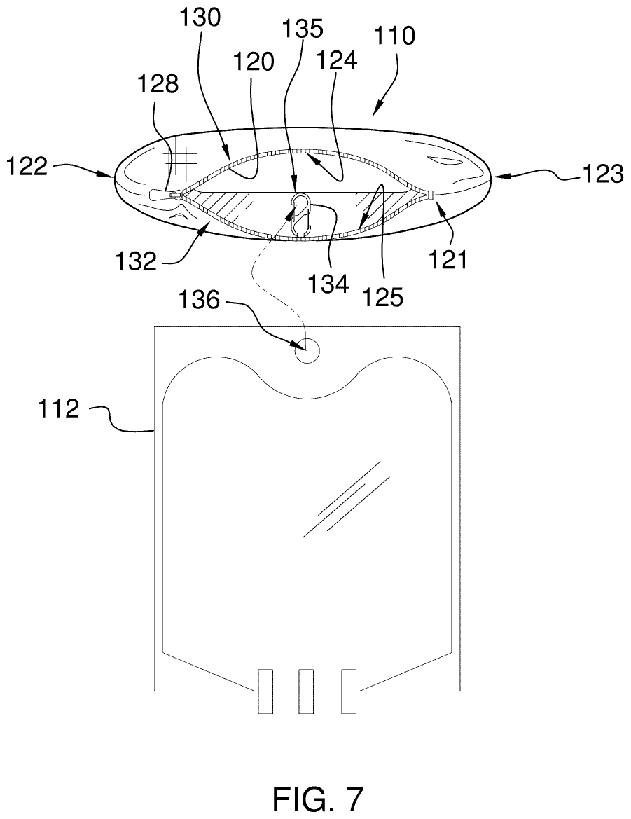

A fifth closure 128 has a first portion 130 which is releasably matable to a second portion 132. The first portion 130 is coupled to the primary edge 124 of the cut 120 in the outside wall 118 of the storage pouch 110. The second portion 132 is coupled to the secondary edge 125 of the cut 120 in the outside wall 118. Additionally, the fifth closure 128 may comprise a zipper or other type of multiple use, mechanical fastener. As is shown in FIG. 7, a clip 134 is installed inside of the storage pouch 110 and the clip 134 has a curved finger 135 which can be through a clip hole 136 in the bag of intravenous medication 112 to secure the bag of intravenous medication 112 in the storage pouch 110.

A plurality of first couplers 138 is each coupled to the front surface 24 of the panel 18. Respective ones of the plurality of first couplers 138 is distributed around the opening 22 in the panel 18. Additionally, respective ones of the plurality of first couplers 138 is located on a respective one of the pair of lobe sections 38 of the perimeter edge 28 of the panel 18. A plurality of second couplers 140 is provided and each second coupler 140 of the plurality of second couplers 140 is coupled to the rear surface 126 of the back wall 116 of the storage pouch 110. Additionally, each second coupler 140 of the plurality of second couplers 140 is releasably matable to a respective first coupler 138 of the plurality of first couplers 138 for removably retaining the storage pouch 110 on the panel 18. Each of the first couplers 138 and the second couplers 140 may comprise respective portions of a hook and loop fastener or other type of multiple use coupler.

In use, the intravenous pump 70 is placed in the storage pocket 68 and the third closure 106 is mated to the second closure 94 to secure the cover 96 over the intravenous pump 70. The bag of intravenous medication 112 is placed into the storage pouch 110 and the storage pouch 110 is attached to the panel 18. Additionally, the intravenous line 115 is routed through the access opening 114 in the storage pouch 110 and the opening 22 in the panel 18. The pair of shoulder straps 44 are worn over the shoulder 40 of the user 16 and the pair of chest straps 58 are mated together to secure the intravenous pump 70 and the bag of intravenous medication 112 on the back 20 of the user 16. Furthermore, the pair of shoulder straps 44 and the pair of chest straps 58 facilitate the user 16 to perform all of their normal daily activities while the intravenous pump 70 is delivering intravenous medication to the user 16. The intravenous line 115 can be secured between the panel 18 and the body 14 of the user 16, if desired, to inhibit the intravenous line 115 from becoming snagged and potentially dislodged while the user 16 is performing their normal daily activities. Furthermore, the body harness 12 and the panel 18 are manufactured with dimensions that are appropriate for ensure a snug fit on young children.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An intravenous pump backpack device for supporting an intravenous pump and a bag of intravenous medication on a user, said device comprising:
a body harness being configured to be worn around a body of the user;
a panel being coupled to said body harness wherein said panel is configured to be supported on a back of the user when the user is wearing said body harness, said panel having an opening extending through said panel;
a storage pocket being coupled to said panel wherein said storage pocket is configured to store the intravenous pump, said storage pocket having a hole being aligned with said opening in said panel wherein said hole is configured to have the intravenous pump be accessible through said opening when the intravenous pump is being stored in said storage pocket; and
a storage pouch being removably attachable to said panel wherein said storage pouch is configured to contain the bag of intravenous medication in proximity to the intravenous pump when said storage pouch is removably attached to said panel, said storage pouch being positionable on an opposite side of said panel from said storage pocket, said storage pouch having an access opening, said access opening being aligned with said opening in said panel when said storage pouch is removably attached to said panel wherein said access opening is configured to expose the bag of intravenous medication to the intravenous pump for connecting the bag of intravenous medication to the intravenous pump.

2. The device according to claim 1, wherein:
said panel has a front surface and a back surface and a perimeter edge extending between said front surface and said back surface;
said perimeter edge has a top side and a first lateral side and a second lateral side and a bottom side;
said perimeter edge has a pair of lobe sections;
each lobe section of said pair of lobe sections extends upwardly from said top side wherein each lobe section of said pair of lobe sections is configured to extend toward a respective shoulder of the user when the user is wearing said backpack;
each lobe section of said pair of lobe sections is aligned with a respective one of said first lateral side and said second lateral side;
each of said first lateral side and said second lateral side curves outwardly between said top side and said bottom side wherein said panel is configured to extend across a full width of the back of the user when the user is wearing said body harness;
said opening extends between said front surface and said back surface; and
said opening is positioned closer to said bottom side than said top side.

3. The device according to claim 2, wherein:
said panel has a pair of flaps;
each flap of said pair of flaps is coupled to and extends laterally away from a respective one of said first lateral side and said second lateral side of said perimeter edge wherein each flap of said pair of flaps is configured to extend around the body of the user when the user is wearing said body harness; and
each flap of said pair of flaps has an upper edge being oriented at an angle with a bottom edge.

4. The device according to claim 3, wherein:
wherein said body harness includes a pair of shoulder straps;
each shoulder strap of said pair of shoulder straps has a first end and a second end;
each shoulder strap of said pair of shoulder straps has a first connection point;
said first connection point of a respective shoulder strap is located adjacent to said first end of said respective shoulder strap;
each shoulder strap of said pair of shoulder straps is attached to a respective lobe section of said pair of lobed sections of said perimeter edge of said panel at said first connection point; and
said second end of each shoulder strap of said pair of shoulder straps is coupled to said upper edge of a respective one of said pair of flaps of said panel.

5. The device according to claim 4, wherein:
said body harness includes a pair of chest straps;
each chest strap of said pair of chest straps has a coupled end and a free end;
said coupled end of said chest strap of said pair of chest straps is movably coupled to a respective one of said pair of shoulder straps wherein each chest strap of said pair of chest straps is configured to extend across a chest of the user when the user is wearing said body harness; and
said free end of said pair of chest straps is releasably matable to each other wherein said pair of chest straps is configured to inhibit said pair of shoulder straps from sliding sideways off of the respective shoulder of the user.

6. The device according to claim 1, wherein:
said storage pocket has a rear wall and a front wall and an outer wall extending between said rear wall and said front wall;
said hole extends through said front wall;
said front wall has a forward surface;
said forward surface is coupled to said back surface of said panel;
said outer wall has a cut extending through said outer wall;
said cut extends around a partial perimeter of said outer wall;
said cut defines a flap portion of said storage pocket;
said flap portion is positionable in a closed position wherein said flap portion is configured to retain the intravenous pump in said storage pocket; and
said flap portion is positionable in an open position wherein said storage pocket is configured to have the intravenous pump being removed from or being installed in said storage pocket.

7. The device according to claim 6, wherein:
said cut has a first edge and a second edge;
said device includes a first closure being coupled to said first edge of said cut in said outer wall of said storage pocket;
said first closure extends along a full length of said first edge;
said device includes a second closure being coupled to said second edge of said cut in said outer wall;
said second closure extends along a full length of said second edge; and
said second closure is releasably matable to said first closure for securing said pocket in said closed position.

8. The device according to claim 7, wherein:
said hole in said front wall of said storage pocket has a bounding edge;
said device includes a third closure being coupled to said bounding edge of said hole in said rear wall of said storage pocket; and
said third closure extends around a partial perimeter of said bounding edge.

9. The device according to claim 8, wherein:
said device includes a cover having a pair of leg portions each extending downwardly from a body portion of said cover;
said lower edge is spaced upwardly from a distal end of each leg portion of said pair of leg portions;
said device includes a fourth closure being coupled to said cover;
said fourth closure extends over said body portion of said cover and downwardly along each leg portion of said pair of leg portions of said cover; and
said fourth closure is releasably matable to said third closure.

10. The device according to claim 9, wherein:
said body portion of said cover has a lower edge; and
said lower edge of said body portion is spaced upwardly from said bounding edge of said hole in said rear wall of said storage pocket when said fourth closure is releasably mated to said third closure wherein said cover is configured to secure the intravenous pump in said storage pocket having controls of the intravenous pump being exposed in said hole.

11. The device according to claim 1, wherein:
said storage pouch has a back wall and an outside wall;
said outside wall has a cut in said outside wall extending into an interior of said storage pouch wherein said cut in said outside wall is configured to pass the bag of intravenous medication into or out of said storage pouch;
said outside wall has an upper side and a first lateral side of said outside wall and a second lateral side of said outside wall; and
said cut in said outside wall extends along a full length of said upper side and downwardly along each of said first lateral side of said outside wall and said second lateral side of said outside wall.

12. The device according to claim 2, wherein:
said device includes a plurality of first couplers;
each first coupler of said plurality of first couplers is coupled to said front surface of said panel;
respective ones of said plurality of first couplers is distributed around said opening in said panel; and
respective ones of said plurality of first couplers is located on a respective one of said pair of lobe sections of said perimeter edge of said panel.

13. The device according to claim 11, wherein:
said device includes a plurality of first couplers;
each first coupler of said plurality of first couplers is coupled to said panel;
said back wall of said storage pouch has a rear surface;
said device includes a plurality of second couplers;
each second coupler of said plurality of second couplers is coupled to said rear surface of said back wall of said storage pouch;
each second coupler of said plurality of second couplers is releasably matable to a respective first coupler of said plurality of first couplers for removably retaining said storage pouch on said panel.

14. The device according to claim 11, wherein:
said device includes a first closure being coupled to said storage pocket;
said device includes a second closure being coupled to said storage pocket;
said device includes a third closure being coupled to said storage pocket;
a fourth closure being coupled to said cover;
said cut in said outside wall of said storage pouch has a primary edge and a secondary edge;
said device includes a fifth closure having a first portion being releasably matable to a second portion;
said first portion is coupled to said primary edge of said cut in said outside wall of said storage pouch; and
said second portion is coupled to said secondary edge of said cut in said outside wall.

15. An intravenous pump backpack device for supporting an intravenous pump and a bag of intravenous medication on a user, said device comprising:
a body harness being configured to be worn around a body of the user;
a panel being coupled to said body harness wherein said panel is configured to be supported on a back of the user when the user is wearing said body harness, said panel having an opening extending through said panel, said panel having a front surface and a back surface and a perimeter edge extending between said front surface and said back surface, said perimeter edge having a top side and a first lateral side and a second lateral side and a bottom side, said perimeter edge having a pair of lobe sections, each lobe section of said pair of lobe sections extending upwardly from said top side wherein each lobe section of said pair of lobe sections is configured to extend toward a respective shoulder of the user when the user is wearing said backpack, each lobe section of said pair of lobe sections being aligned with a respective one of said first lateral side and said second lateral side, each of said first lateral side and said second lateral side curving outwardly between said top side and said bottom side wherein said panel is configured to extend across a full width of the back of the user when the user is wearing said body harness, said opening extending between said front surface and said back surface, said opening being positioned closer to said bottom side than said top side, said panel having a pair of flaps, each flap of said pair of flaps being coupled to and extending laterally away from a respective one of said first lateral side and said second lateral side of said perimeter edge wherein each flap of said pair of flaps is configured to extend around the body of the user when the user is wearing said body harness, each flap of said pair of flaps having an upper edge being oriented at an angle with a bottom edge;
wherein said body harness includes a pair of shoulder straps, each shoulder strap of said pair of shoulder straps having a first end and a second end, each shoulder strap of said pair of shoulder straps having a first connection point, said first connection point of a respective shoulder strap being located adjacent to said first end of said respective shoulder strap, each shoulder strap of said pair of shoulder straps being attached to a respective lobe section of said pair of lobed sections of said perimeter edge of said panel at said first connection point, said second end of each shoulder strap of said pair of shoulder straps being coupled to said upper edge of a respective one of said pair of flaps of said panel;
wherein said body harness includes a pair of chest straps, each chest strap of said pair of chest straps having a coupled end and a free end, said coupled end of said chest strap of said pair of chest straps being movably coupled to a respective one of said pair of shoulder straps wherein each chest strap of said pair of chest straps is configured to extend across a chest of the user when the user is wearing said body harness, said free end of said pair of chest straps being releasably matable to each other wherein said pair of chest straps is configured to inhibit said pair of shoulder straps from sliding sideways off of the respective shoulder of the user;
a storage pocket being coupled to said panel wherein said storage pocket is configured to store the intravenous pump, said storage pocket having a hole being aligned with said opening in said panel wherein said hole is configured to have the intravenous pump be accessible through said opening when the intravenous pump is being stored in said storage pocket, said storage pocket having a rear wall and a front wall and an outer wall extending between said rear wall and said front wall, said hole extending through said front wall, said hole having a bounding edge, said front wall having a forward surface, said forward surface being coupled to said back surface of said panel, said outer wall having a cut extending through said outer wall, said cut extending around a partial perimeter of said outer wall, said cut defining a flap portion of said storage pocket, said cut having a first edge and a second edge, said flap portion being positionable in a closed position wherein said flap portion is configured to retain the intravenous pump in said storage pocket, said flap portion being positionable in an open position wherein said storage pocket is configured to have the intravenous pump being removed from or being installed in said storage pocket;
a first closure being coupled to said first edge of said cut in said outer wall of said storage pocket, said first closure extending along a full length of said first edge;
a second closure being coupled to said second edge of said cut in said outer wall, said second closure extending along a full length of said second edge, said second closure being releasably matable to said first closure for securing said pocket in said closed position;
a cover having a pair of leg portions each extending downwardly from a body portion of said cover, said body portion having a lower edge, said lower edge being spaced upwardly from a distal end of each leg portion of said pair of leg portions;
a third closure being coupled to said bounding edge of said hole in said front wall of said storage pocket, said third closure extending around a partial perimeter of said bounding edge;
a fourth closure being coupled to said cover, said fourth closure extending over said body portion of said cover and downwardly along each leg portion of said pair of leg portions of said cover, said fourth closure being releasably matable to said third closure, said lower edge of said body portion being spaced upwardly from said bounding edge of said hole in said rear wall of said storage pocket when said fourth closure is releasably mated to said third closure wherein said cover is configured to secure the intravenous pump in said storage pocket having controls of the intravenous pump being exposed in said hole;
a storage pouch being removably attachable to said panel wherein said storage pouch is configured to contain the bag of intravenous medication in proximity to the intravenous pump when said storage pouch is removably attached to said panel, said storage pouch being positionable on an opposite side of said panel from said storage pocket, said storage pouch having an access opening, said access opening being aligned with said opening in said panel when said storage pouch is removably attached to said panel wherein said access opening is configured to expose the bag of intravenous medication to the intravenous pump for connecting the bag of intravenous medication to the intravenous pump, said storage pouch having a back wall and an outside wall, said outside wall having a cut in said outside wall extending into an interior of said storage pouch wherein said cut in said outside wall is configured to pass the bag of intravenous medication into or out of said storage pouch, said outside wall having an upper side and a first lateral side of said outside wall and a second lateral side of said outside wall, said cut in said outside wall extending along a full length of said upper side and downwardly along each of said first lateral side of said outside wall and said second lateral side of said outside wall, said cut in said outside wall having a primary edge and a secondary edge, said back wall having a rear surface;
a fifth closure having a first portion being releasably matable to a second portion, said first portion being coupled to said primary edge of said cut in said outside wall of said storage pouch, said second portion being coupled to said secondary edge of said cut in said outside wall;
a plurality of first couplers, each first coupler of said plurality of first couplers being coupled to said front surface of said panel, respective ones of said plurality of first couplers being distributed around said opening in said panel, respective ones of said plurality of first couplers being located on a respective one of said pair of lobe sections of said perimeter edge of said panel; and
a plurality of second couplers, each second coupler of said plurality of second couplers being coupled to said rear surface of said back wall of said storage pouch, each second coupler of said plurality of second couplers being releasably matable to a respective first coupler of said plurality of first couplers for removably retaining said storage pouch on said panel.
